(19) United States
(12) Patent Application Publication
(10) Pub. No.: US 2021/0346451 A1
(43) Pub. Date: Nov. 11, 2021

(54) CHINESE MEDICINE COMPOSITION AND PREPARATION METHOD AND USE THEREOF

(71) Applicant: WEFINE HEALTH TECHNOLOGY CORPORATION, SAN JOSE, CA (US)

(72) Inventors: Jiyuan LIU, Changxing (CN); Huyun DONG, Santa Clara, CA (US); Yili HU, San Jose, CA (US); Xiangqiu LIU, Changxing (CN)

(21) Appl. No.: 16/870,997

(22) Filed: May 10, 2020

(51) Int. Cl. A61K 36/481; A61K 36/074; A61K 36/258; A61K 36/355; A61K 36/71; A61K 36/315; A61K 36/53; A61K 36/634; A61K 36/752; A61K 36/704; A61K 36/236

(52) U.S. Cl. CPC A61K 36/481; A61K 36/074; A61K 36/258; A61K 36/355; A61K 36/71; A61K 36/236; A61K 36/53; A61K 36/634; A61K 36/752; A61K 36/704; A61K 36/315

(57) ABSTRACT

Technologies relating to Chinese medicine composition used for treatment of protecting respiratory tract from viral infection by significantly increasing immunity of patients are disclosed. An example composition used for treatment of protecting respiratory tract from viral infection by significantly increasing immunity of a patient, includes: an Astragalus Root; a Ganoderma Spore; a Siberian Ginseng; an Underleaf Pearl; a Honeysuckle Flower; a Chinese Globe Flower; an Isatis Leaf; an Woad Root; a Patchouli; a Forsythia Fruit; an asiatic Pennywort; an Aged Tangerine Peel; a Giant Knotweed; a Reed Rhizome; an Unprepared Licorice Root; and a Fresh Ginger Rhizome.

### TECHNICAL FIELD

The present disclosure relates generally to Chinese medicine compositions and preparation method and use thereof, and more specifically to Chinese medicine composition used for the treatment of protecting the respiratory tract from viral infection by significantly increasing the immunity of patients.

### BACKGROUND

Viral respiratory disease is a highly contagious disease that seriously endangers human health. According to incomplete statistics, about 75% of pandemic infections are caused by viral infections. In recent years, virus genome variation and the emergence of new virus strains, such as SARS in China in 2003, H1N1 in 2009, H7N9 in 2013, COVID-19 in many countries around the world in 2019 to date, have caused great harm and death to human beings. The continued emergence of new virus strains and virus variation and re-emergence of old infectious diseases bring unprecedented challenges for human prevention and treatment. Effective drug and vaccine research continues to lag. Therefore, how to improve the human body's immunity, prevent the infection of such infectious diseases, or protect vulnerable people, is of great significance, from the perspective of either individual health status or socio-economic development.

The solutions to the existing defects in the prevention of the infectious virus, virus genome variation, and new virus strains are vital in response to global pandemics. It is therefore highly desirable to provide a pure Chinese herbal medicine composition with significantly improved immunity and disease resistance in people to protect their respiratory tract from viral infection without other serious side effects.

### SUMMARY

Technologies relating to Chinese medicine composition used for the treatment of protecting the respiratory tract from viral infection by significantly increasing immunity of patients are disclosed.

In a 9-ingredients implementations of the present disclosure, the composition includes an Astragalus Root in an amount from 5 units to 50 units by weight with respect to a total weight of the composition, a Honeysuckle Flower in an amount from 5 units to 50 units by weight with respect to the total weight of the composition, an Isatis Leaf in an amount from 5 units to 30 units by weight with respect to the total weight of the composition, an Woad Root in an amount from 6 units to 50 units by weight with respect to the total weight of the composition, a Patchouli in an amount from 6 units to 30 units by weight with respect to the total weight of the composition, a Forsythia Fruit in an amount from 6 units to 30 units by weight with respect to the total weight of the composition, an Aged Tangerine Peel in an amount from 3 units to 30 units by weight with respect to the total weight of the composition, a Reed Rhizome in an amount from 9 units to 60 units by weight with respect to the total weight of the composition, and an Unprepared Licorice Root in an amount from 2 units to 10 units by weight with respect to the total weight of the composition.

To be more specific, in the 9-ingredients implementations of the present disclosure, the composition includes an Astragalus Root in an amount of 9 units by weight with respect to a total weight of the composition, a Honeysuckle Flower in an amount of 5 units by weight with respect to the total weight of the composition, an Isatis Leaf in an amount of 5 units by weight with respect to the total weight of the composition, an Woad Root in an amount of 5 units by weight with respect to the total weight of the composition, a Patchouli in an amount of 6 units by weight with respect to the total weight of the composition, a Forsythia Fruit in an amount of 5 units by weight with respect to the total weight of the composition, an Aged Tangerine Peel in an amount of 3 units by weight with respect to the total weight of the composition, a Reed Rhizome in an amount of 9 units by weight with respect to the total weight of the composition, and an Unprepared Licorice Root in an amount of 2 units by weight with respect to the total weight of the composition.

In a 10-ingredients implementations of the present disclosure, the composition includes an Astragalus Root in an amount from 5 units to 50 units by weight with respect to a total weight of the composition, a Honeysuckle Flower in an amount from 5 units to 50 units by weight with respect to the total weight of the composition, an Woad Root in an amount from 6 units to 50 units by weight with respect to the total weight of the composition, a Patchouli in an amount from 6 units to 30 units by weight with respect to the total weight of the composition, a Forsythia Fruit in an amount from 6 units to 30 units by weight with respect to the total weight of the composition, an Aged Tangerine Peel in an amount from 3 units to 30 units by weight with respect to the total weight of the composition, a Giant Knotweed in an amount from 6 units to 50 units by weight with respect to the total weight of the composition, a Reed Rhizome in an amount from 9 units to 60 units by weight with respect to the total weight of the composition, an Unprepared Licorice Root in an amount from 2 units to 10 units by weight with respect to the total weight of the composition, and a Fresh Ginger Rhizome in an amount from 2 units to 15 units by weight with respect to the total weight of the composition.

To be more specific, in the 10-ingredients implementations of the present disclosure, the composition includes an Astragalus Root in an amount of 9 units by weight with respect to a total weight of the composition, a Honeysuckle Flower in an amount of 6 units by weight with respect to the total weight of the composition, an Woad Root in an amount of 8 units by weight with respect to the total weight of the composition, a Patchouli in an amount of 6 units by weight with respect to the total weight of the composition, a Forsythia Fruit in an amount of 6 units by weight with respect to the total weight of the composition, an Aged Tangerine Peel in an amount of 6 units by weight with respect to the total weight of the composition, a Giant Knotweed in an amount of 5 units by weight with respect to the total weight of the composition, a Reed Rhizome in an amount of 8 units by weight with respect to the total weight of the composition, an Unprepared Licorice Root in an amount of 3 units by weight with respect to the total weight of the composition, and a Fresh Ginger Rhizome in an amount of 3 units by weight with respect to the total weight of the composition.

In a 12-ingredients implementations of the present disclosure, the composition includes an Astragalus Root in an amount from 5 units to 50 units by weight with respect to a total weight of the composition, a Ganoderma Spore in an amount from 2 units to 21 units by weight with respect to the total weight of the composition, a Siberian Ginseng in an amount from 6 units to 30 units by weight with respect to the total weight of the composition, an Underleaf Pearl in an amount from 5 units to 35 units by weight with respect to the total weight of the composition, a Honeysuckle Flower in an amount from 5 units to 50 units by weight with respect to the total weight of the composition, an Woad Root in an amount from 6 units to 50 units by weight with respect to the total weight of the composition, a Patchouli in an amount from 6 units to 30 units by weight with respect to the total weight of the composition, a Forsythia Fruit in an amount from 6 units to 30 units by weight with respect to the total weight of the composition, an Aged Tangerine Peel in an amount from 3 units to 30 units by weight with respect to the total weight of the composition, a Reed Rhizome in an amount from 8 units to 60 units by weight with respect to the total weight of the composition, an Unprepared Licorice Root in an amount from 2 units to 10 units by weight with respect to the total weight of the composition, and a Fresh Ginger Rhizome in an amount from 2 units to 15 units by weight with respect to the total weight of the composition.

To be more specific, in the 12-ingredients implementations of the present disclosure, the composition includes an Astragalus Root in an amount of 9 units by weight with respect to a total weight of the composition, a Ganoderma Spore in an amount of 2 units by weight with respect to the total weight of the composition, a Siberian Ginseng in an amount of 6 units by weight with respect to the total weight of the composition, an Underleaf Pearl in an amount of 6 units by weight with respect to the total weight of the composition, a Honeysuckle Flower in an amount of 6 units by weight with respect to the total weight of the composition, an Woad Root in an amount of 8 units by weight with respect to the total weight of the composition, a Patchouli in an amount of 6 units by weight with respect to the total weight of the composition, a Forsythia Fruit in an amount of 6 units by weight with respect to the total weight of the composition, an Aged Tangerine Peel in an amount of 6 units by weight with respect to the total weight of the composition, a Reed Rhizome in an amount of 8 units by weight with respect to the total weight of the composition, an Unprepared Licorice Root in an amount of 3 units by weight with respect to the total weight of the composition, and a Fresh Ginger Rhizome in an amount of 3 units by weight with respect to the total weight of the composition.

In a 16-ingredient composition of the present disclosure, the composition includes an Astragalus Root in an amount from 5 units to 50 units by weight with respect to a total weight of the composition, a Ganoderma Spore in an amount from 2 units to 21 units by weight with respect to the total weight of the composition, a Siberian Ginseng in an amount from 6 units to 30 units by weight with respect to the total weight of the composition, an Underleaf Pearl in an amount from 5 units to 35 units by weight with respect to the total weight of the composition, a Honeysuckle Flower in an amount from 5 units to 50 units by weight with respect to the total weight of the composition, a Chinese Globe Flower in an amount from 5 units to 30 units by weight with respect to the total weight of the composition, an Isatis Leaf in an amount from 5 units to 30 units by weight with respect to the total weight of the composition, an Woad Root in an amount from 6 units to 50 units by weight with respect to the total weight of the composition, a Patchouli in an amount from 6 units to 30 units by weight with respect to the total weight of the composition, a Forsythia Fruit in an amount from 6 units to 30 units by weight with respect to the total weight of the composition, an asiatic Pennywort in an amount from 5 units to 50 units by weight with respect to the total weight of the composition, an Aged Tangerine Peel in an amount from 3 units to 30 units by weight with respect to the total weight of the composition, a Giant Knotweed in an amount from 6 units to 50 units by weight with respect to the total weight of the composition, a Reed Rhizome in an amount from 9 units to 60 units by weight with respect to the total weight of the composition, an Unprepared Licorice Root in an amount from 2 units to 10 units by weight with respect to the total weight of the composition, and a Fresh Ginger Rhizome in an amount from 2 units to 15 units by weight with respect to the total weight of the composition.

To be more specific, in the 16-ingredient composition of the present disclosure, the composition includes an Astragalus Root in an amount of 15 units by weight with respect to a total weight of the composition, a Ganoderma Spore in an amount of 3 units by weight with respect to the total weight of the composition, a Siberian Ginseng in an amount of 10 units by weight with respect to the total weight of the composition, an Underleaf Pearl in an amount of 10 units by weight with respect to the total weight of the composition, a Honeysuckle Flower in an amount of 12 units by weight with respect to the total weight of the composition, a Chinese Globe Flower in an amount of 10 units by weight with respect to the total weight of the composition, an Isatis Leaf in an amount of 10 units by weight with respect to the total weight of the composition, an Woad Root in an amount of 10 units by weight with respect to the total weight of the composition, a Patchouli in an amount of 10 units by weight with respect to the total weight of the composition, a Forsythia Fruit in an amount of 10 units by weight with respect to the total weight of the composition, an asiatic Pennywort in an amount of 10 units by weight with respect to the total weight of the composition, an Aged Tangerine Peel in an amount of 10 units by weight with respect to the total weight of the composition, a Giant Knotweed in an amount of 15 units by weight with respect to the total weight of the composition, a Reed Rhizome in an amount of 15 units by weight with respect to the total weight of the composition, an Unprepared Licorice Root in an amount of 3 units by weight with respect to the total weight of the composition, and a Fresh Ginger Rhizome in an amount of 3 units by weight with respect to the total weight of the composition.

### DETAILED DESCRIPTION

Technologies relating to Chinese medicine composition used for the treatment of protecting the respiratory tract from viral infection by significantly increasing immunity of patients are disclosed. The technologies described in the present disclosure may provide the following technical advantages.

First, the disclosed technology provides pure Chinese herbal intake compositions that may significantly improve human immunity to protect the respiratory tract from infection. Its composition includes Astragalus Root, Ganoderma Spore, Siberian Ginseng, Underleaf Pearl, Honeysuckle Flower, Chinese Globe Flower, Isatis Leaf, Woad Root, Patchouli, Forsythia Fruit, asiatic Pennywort, Aged Tangerine Peel, Giant Knotweed, Reed Rhizome, Unprepared Licorice Root, and Fresh Ginger Rhizome. The granules made of drugs described in the present disclosure have a prominent effect on the prevention of viral respiratory diseases It may improve human immunity, resist the invasion of the respiratory tract by viruses, and play a role in protecting the respiratory mucous membrane barrier. The present disclosure may be used for influenza and other viral respiratory disease or pandemic. It may provide outbreak prevention and protection of the vulnerable population, and reduce the symptoms of infected people. It is a new type of composition, a broad-spectrum preventive medicine used for epidemic prevention, anti-virus protection. The disclosed composition has significant meaning for mass respiratory infectious disease control.

More explicitly, the disclosed technology includes ingredients used to increase the number of polynuclear white blood cells and white blood cells, promote lymphocytes, macrophages, neutrophils activation, and improve the phagocytosis of macrophages. Also, the disclosed technology includes ingredients having antiviral substances used for upper respiratory tract infection treatment.

Finally, because the compositions in the present disclosure are specially designed for vulnerable people such as children, pregnant women, elderly people, malnourished people, people who are ill or immunocompromised, or people who have chronic diseases, the compositions have a lower dose of herbal ingredients than their standard usage. This ensures that it can be implemented as preventive medicine for a broad range of people.

All the results are tested under clinical experiments combined with the basic theory of traditional Chinese medicine including the concept of organic wholeness and the treatment based on syndrome differentiation.

Compositions:

In a 9-ingredients implementations of the present disclosure, the composition includes an Astragalus Root in an amount from 5 units to 50 units by weight with respect to a total weight of the composition, a Honeysuckle Flower in an amount from 5 units to 50 units by weight with respect to the total weight of the composition, an Isatis Leaf in an amount from 5 units to 30 units by weight with respect to the total weight of the composition, an Woad Root in an amount from 6 units to 50 units by weight with respect to the total weight of the composition, a Patchouli in an amount from 6 units to 30 units by weight with respect to the total weight of the composition, a Forsythia Fruit in an amount from 6 units to 30 units by weight with respect to the total weight of the composition, an Aged Tangerine Peel in an amount from 3 units to 30 units by weight with respect to the total weight of the composition, a Reed Rhizome in an amount from 9 units to 60 units by weight with respect to the total weight of the composition, and an Unprepared Licorice Root in an amount from 2 units to 10 units by weight with respect to the total weight of the composition.

To be more specific, in the 9-ingredients implementations of the present disclosure, the composition includes an Astragalus Root in an amount of 9 units by weight with respect to a total weight of the composition, a Honeysuckle Flower in an amount of 5 units by weight with respect to the total weight of the composition, an Isatis Leaf in an amount of 5 units by weight with respect to the total weight of the composition, an Woad Root in an amount of 5 units by weight with respect to the total weight of the composition, a Patchouli in an amount of 6 units by weight with respect to the total weight of the composition, a Forsythia Fruit in an amount of 5 units by weight with respect to the total weight of the composition, an Aged Tangerine Peel in an amount of 3 units by weight with respect to the total weight of the composition, a Reed Rhizome in an amount of 9 units by weight with respect to the total weight of the composition, and an Unprepared Licorice Root in an amount of 2 units by weight with respect to the total weight of the composition.

In a 10-ingredients implementations of the present disclosure, the composition includes an Astragalus Root in an amount from 5 units to 50 units by weight with respect to a total weight of the composition, a Honeysuckle Flower in an amount from 5 units to 50 units by weight with respect to the total weight of the composition, an Woad Root in an amount from 6 units to 50 units by weight with respect to the total weight of the composition, a Patchouli in an amount from 6 units to 30 units by weight with respect to the total weight of the composition, a Forsythia Fruit in an amount from 6 units to 30 units by weight with respect to the total weight of the composition, an Aged Tangerine Peel in an amount from 3 units to 30 units by weight with respect to the total weight of the composition, a Giant Knotweed in an amount from 6 units to 50 units by weight with respect to the total weight of the composition, a Reed Rhizome in an amount from 9 units to 60 units by weight with respect to the total weight of the composition, an Unprepared Licorice Root in an amount from 2 units to 10 units by weight with respect to the total weight of the composition, and a Fresh Ginger Rhizome in an amount from 2 units to 15 units by weight with respect to the total weight of the composition.

To be more specific, in the 10-ingredients implementations of the present disclosure, the composition includes an Astragalus Root in an amount of 9 units by weight with respect to a total weight of the composition, a Honeysuckle Flower in an amount of 6 units by weight with respect to the total weight of the composition, an Woad Root in an amount of 8 units by weight with respect to the total weight of the composition, a Patchouli in an amount of 6 units by weight with respect to the total weight of the composition, a Forsythia Fruit in an amount of 6 units by weight with respect to the total weight of the composition, an Aged Tangerine Peel in an amount of 6 units by weight with respect to the total weight of the composition, a Giant Knotweed in an amount of 5 units by weight with respect to the total weight of the composition, a Reed Rhizome in an amount of 8 units by weight with respect to the total weight of the composition, an Unprepared Licorice Root in an amount of 3 units by weight with respect to the total weight of the composition, and a Fresh Ginger Rhizome in an amount of 3 units by weight with respect to the total weight of the composition.

In a 12-ingredients implementations of the present disclosure, the composition includes an Astragalus Root in an amount from 5 units to 50 units by weight with respect to a total weight of the composition, a Ganoderma Spore in an amount from 2 units to 21 units by weight with respect to the total weight of the composition, a Siberian Ginseng in an amount from 6 units to 30 units by weight with respect to the total weight of the composition, an Underleaf Pearl in an amount from 5 units to 35 units by weight with respect to the total weight of the composition, a Honeysuckle Flower in an amount from 5 units to 50 units by weight with respect to the total weight of the composition, an Woad Root in an amount from 6 units to 50 units by weight with respect to the total weight of the composition, a Patchouli in an amount from 6 units to 30 units by weight with respect to the total weight of the composition, a Forsythia Fruit in an amount from 6 units to 30 units by weight with respect to the total weight of the composition, an Aged Tangerine Peel in an amount from 3 units to 30 units by weight with respect to the total weight of the composition, a Reed Rhizome in an amount from 8 units to 60 units by weight with respect to the total weight of the composition, an Unprepared Licorice Root in an amount from 2 units to 10 units by weight with respect to the total weight of the composition, and a Fresh Ginger Rhizome in an amount from 2 units to 15 units by weight with respect to the total weight of the composition.

To be more specific, in the 12-ingredients implementations of the present disclosure, the composition includes an Astragalus Root in an amount of 9 units by weight with respect to a total weight of the composition, a Ganoderma Spore in an amount of 2 units by weight with respect to the total weight of the composition, a Siberian Ginseng in an amount of 6 units by weight with respect to the total weight of the composition, an Underleaf Pearl in an amount of 6 units by weight with respect to the total weight of the composition, a Honeysuckle Flower in an amount of 6 units by weight with respect to the total weight of the composition, an Woad Root in an amount of 8 units by weight with respect to the total weight of the composition, a Patchouli in an amount of 6 units by weight with respect to the total weight of the composition, a Forsythia Fruit in an amount of 6 units by weight with respect to the total weight of the composition, an Aged Tangerine Peel in an amount of 6 units by weight with respect to the total weight of the composition, a Reed Rhizome in an amount of 8 units by weight with respect to the total weight of the composition, an Unprepared Licorice Root in an amount of 3 units by weight with respect to the total weight of the composition, and a Fresh Ginger Rhizome in an amount of 3 units by weight with respect to the total weight of the composition.

In a 16-ingredient composition of the present disclosure, the composition includes an Astragalus Root in an amount from 5 units to 50 units by weight with respect to a total weight of the composition, a Ganoderma Spore in an amount from 2 units to 21 units by weight with respect to the total weight of the composition, a Siberian Ginseng in an amount from 6 units to 30 units by weight with respect to the total weight of the composition, an Underleaf Pearl in an amount from 5 units to 35 units by weight with respect to the total weight of the composition, a Honeysuckle Flower in an amount from 5 units to 50 units by weight with respect to the total weight of the composition, a Chinese Globe Flower in an amount from 5 units to 30 units by weight with respect to the total weight of the composition, an Isatis Leaf in an amount from 5 units to 30 units by weight with respect to the total weight of the composition, an Woad Root in an amount from 6 units to 50 units by weight with respect to the total weight of the composition, a Patchouli in an amount from 6 units to 30 units by weight with respect to the total weight of the composition, a Forsythia Fruit in an amount from 6 units to 30 units by weight with respect to the total weight of the composition, an asiatic Pennywort in an amount from 5 units to 50 units by weight with respect to the total weight of the composition, an Aged Tangerine Peel in an amount from 3 units to 30 units by weight with respect to the total weight of the composition, a Giant Knotweed in an amount from 6 units to 50 units by weight with respect to the total weight of the composition, a Reed Rhizome in an amount from 9 units to 60 units by weight with respect to the total weight of the composition, an Unprepared Licorice Root in an amount from 2 units to 10 units by weight with respect to the total weight of the composition, and a Fresh Ginger Rhizome in an amount from 2 units to 15 units by weight with respect to the total weight of the composition.

To be more specific, in the 16-ingredient composition of the present disclosure, the composition includes an Astragalus Root in an amount of 15 units by weight with respect to a total weight of the composition, a Ganoderma Spore in an amount of 3 units by weight with respect to the total weight of the composition, a Siberian Ginseng in an amount of 10 units by weight with respect to the total weight of the composition, an Underleaf Pearl in an amount of 10 units by weight with respect to the total weight of the composition, a Honeysuckle Flower in an amount of 12 units by weight with respect to the total weight of the composition, a Chinese Globe Flower in an amount of 10 units by weight with respect to the total weight of the composition, an Isatis Leaf in an amount of 10 units by weight with respect to the total weight of the composition, an Woad Root in an amount of 10 units by weight with respect to the total weight of the composition, a Patchouli in an amount of 10 units by weight with respect to the total weight of the composition, a Forsythia Fruit in an amount of 10 units by weight with respect to the total weight of the composition, an asiatic Pennywort in an amount of 10 units by weight with respect to the total weight of the composition, an Aged Tangerine Peel in an amount of 10 units by weight with respect to the total weight of the composition, a Giant Knotweed in an amount of 15 units by weight with respect to the total weight of the composition, a Reed Rhizome in an amount of 15 units by weight with respect to the total weight of the composition, an Unprepared Licorice Root in an amount of 3 units by weight with respect to the total weight of the composition, and a Fresh Ginger Rhizome in an amount of 3 units by weight with respect to the total weight of the composition.

It is noted that the term “unit” referred to hereinabove and hereinafter is intended to mean any mass unit such as gram (g), kilogram (kg), ounce (oz), pound (lb), etc.

The production process of the Chinese medicine composition in the present disclosure may be extracted with conventional extraction methods. It can be produced as capsules, tablets, granulated punches, and tea bags.

Each Ingredient in the Compositions:

Astragalus Root: Also known as Astragalus Radix. Chinese medicine considers that Astragalus Root is capable of invigorating qi for consolidating superficies, strengthening the spleen and tonifying the lung, and expelling the pathogenic factor. It is an essential medicine for supplementing the body's deficiency. It is also capable of protecting the healthy qi of the spleen and lung, reinforcing the vital essence and strengthening the primordial qi, and keeping out the pathogenic factors. According to modern pharmacological research, the main chemical components of Astragalus Root are Astragalus Polysacharins (APS), flavonoids, saponins, etc. These components can regulate the body's immune function. They may not only increase the number of polynuclear white blood cells and white blood cells, but also promote the activation of lymphocytes, macrophages, and neutrophils, improve the phagocytosis of macrophages, and accelerate the response to invasive pathogens. Astragalus Root is generally an immune enhancer.

Ganoderma Spore: Also known as Spore Powder of Ganoderma lucidum, or Spore Powder of lingzhi Mushroom (or leishi Mushroom). Ganoderma Spores are extremely small spores ejected from the cover during the mature stage of lingzhi growth, and are the reproductive cells of lingzhi, which have all the genetic active substances of lingzhi. Ganoderma Spore contains rich polysaccharide, and thus may enhance the mononuclear phagocytosis system, body fluids, and cell immunity. It has a significant immune regulation function.

Siberian Ginseng: Also known as Eleutherococci Senticosi Radix Et Caulis. Chinese medicine considers that it is capable of supplementing the center of the body and boosting the qi, and strengthening bones and willpower. According to modern pharmacological research, the main chemical components of Siberian Ginseng contains Acanthopanax senticosus Saponins extract, flavonoids, polysaccharide, etc. These components may regulate human immune function, protect the heart, brain, and liver, and enhance the body's ability to resist adverse stimuli.

Underleaf Pearl: Also known as Phyllanthus urinaria. Chinese medicine considers that it is capable of soothing the liver, antipyretic, inducing diuresis, and detoxifying. Pharmacological studies consider that it has antiviral, disease-resistant microorganisms, and antioxidant effect.

Honeysuckle Flower: Also known as lonicerae Flos. Chinese medicine considers that Honeysuckle Flower has a strong detoxification function. Pharmacological studies prove that the main chemical components of Honeysuckle Flower contain volatile oil, flavonoids, triamcinolone, etc. These components have a prominent inhabitation effect against the influenza virus and other viruses.

Chinese Globe Flower: Also known as Trollius Chinensis Bunge. Pharmacological studies prove that the main chemical components of Chinese Globe Flower are mainly flavonoids, organic acids, alkaloids, etc. It has antiviral, antioxidant, and anti-bacterial function.

Isatis Leaf, and Woad Root: Isatis Leaf is also known as isatidis folium. Woad Root is also known as isatidis Radix. These two Chinese medicines are considered to have the function of detoxifying heat, cooling blood, and relieving sore throat. Many pharmacological studies have shown that they have a broad spectrum of antimicrobial or antiviral functions of bioactive substances. Isatis Leaf has an immune-boosting function.

Patchouli: Also known as Pogostemonis herba. It is capable of resolving dampness with aromatics, relieving stomachache and nausea, and dispelling summer-heat to relieve superficies syndrome. It contains Patchouli Alcohol, flavonoids, and alkaloids which have the function of inhibiting the proliferation of viruses, and have an immune regulation function. It may regulate gastrointestinal movement, and protect the intestinal barrier.

Forsythia Fruit: Also known as Forsythiae fructus. It has the function of reducing heat and detoxifying, and removing swelling and lump. It contains Forsythoside, which has antiviral, regulating immunity, and anti-heat function.

Asiatic Pennywort: Also known as Centellae herba. Chinese medicine considers that it is capable of removing heat and wet, detoxifying and reducing swelling function. Asiatic Pennywort contains flavonoids which have a strong antioxidant effect and an immune system regulating function.

Aged Tangerine Peel: Also known as Citri reticulatae pericarpium. Chinese medicine considers that it is capable of strengthening spleen and improving qi, drying wet interior, and moistening the lungs and eliminating the phlegm. Modern pharmacological studies have shown that the Aged Tangerine Peel contains a variety of vitamin B, vitamin C, and flavonoids. It has anti-inflammatory and regulating immune function.

Giant Knotweed: Also known as polygoni cuspidati rhizoma. Chinese medicine considers that it is capable of eliminating dampness and curing jaundice, clearing heat and resolving toxin, dissipating blood stasis and relieving pain, and relieving cough and reducing sputum. Its extract and its main compounds include antioxidants, anti-infection and other functions. Several studies have been shown that it is capable of protecting the function of the liver and lungs. It can also resist pulmonary fibrosis and pulmonary stomatitis.

Reed Rhizome: Also known as Phragmitis rhizoma. Chinese medicine considers that it may clear heat, promote the secretion of saliva, prevent vomiting, and induce diuresis. The main chemical components of Reed Rhizome are polysaccharides and flavonoids which have a certain antioxidant and antibacterial function.

Unprepared Licorice Root: Also known as Radix Glyxyrrhizae uralensis. It contains licorice saponins, licorice polysaccharides, and licorice flavonoids. It has the function of anti-inflammatory, sterilizing, anti-fibrosis, and enhancing the immune system.

Fresh Ginger Rhizome: Also known as Zingiberis rhizoma recens. Chinese medicine considers that it has the function of relieving the exterior of the body and facilitating the lung. It is rich in flavonoids, steroid volatile oil, curcumin, etc. It has a good ability to promote digestion, improve blood circulation, inhibit inflammatory bacteria, inhibit oxidation, and enhance body fluid immunity.

Implementations of 16-Ingredients Composition:

### Example 1

In one implementation, the composition in the present disclosure includes an Astragalus Root in an amount of 25 units by weight with respect to a total weight of the composition, a Ganoderma Spore in an amount of 5 units by weight with respect to the total weight of the composition, a Siberian Ginseng in an amount of 15 units by weight with respect to the total weight of the composition, an Underleaf Pearl in an amount of 12 units by weight with respect to the total weight of the composition, a Honeysuckle Flower in an amount of 15 units by weight with respect to the total weight of the composition, a Chinese Globe Flower in an amount of 10 units by weight with respect to the total weight of the composition, an Isatis Leaf in an amount of 10 units by weight with respect to the total weight of the composition, an Isatis Root in an amount of 15 units by weight with respect to the total weight of the composition, a Patchouli in an amount of 10 units by weight with respect to the total weight of the composition, a Forsythia Fruit in an amount of 10 units by weight with respect to the total weight of the composition, an asiatic Pennywort in an amount of 10 units by weight with respect to the total weight of the composition, an Aged Tangerine Peel in an amount of 10 units by weight with respect to the total weight of the composition, a Giant Knotweed in an amount of 15 units by weight with respect to the total weight of the composition, a Reed Rhizome in an amount of 15 units by weight with respect to the total weight of the composition, an Unprepared Licorice Root in an amount of 6 units by weight with respect to the total weight of the composition, and a Fresh Ginger Rhizome in an amount of 6 units by weight with respect to the total weight of the composition. In the present implementation, the above-mentioned proportioned dose is a daily dose, and it should be taken half an hour before the meal in the morning and evening. The duration of usage is 10 days. In this implementation, it is used to increase the immune function and protect the respiratory tract from virus invasion. It is suitable for the prevention of people over 60 years old or with basic diseases.

### Example 2

In another implementation, the composition in the present disclosure includes an Astragalus Root in an amount of 10 units by weight with respect to a total weight of the composition, a Ganoderma Spore in an amount of 3 units by weight with respect to the total weight of the composition, a Siberian Ginseng in an amount of 10 units by weight with respect to the total weight of the composition, an Underleaf Pearl in an amount of 12 units by weight with respect to the total weight of the composition, a Honeysuckle Flower in an amount of 18 units by weight with respect to the total weight of the composition, a Chinese Globe Flower in an amount of 15 units by weight with respect to the total weight of the composition, an Isatis Leaf in an amount of 18 units by weight with respect to the total weight of the composition, an Isatis Root in an amount of 30 units by weight with respect to the total weight of the composition, a Patchouli in an amount of 12 units by weight with respect to the total weight of the composition, a Forsythia Fruit in an amount of 12 units by weight with respect to the total weight of the composition, an asiatic Pennywort in an amount of 12 units by weight with respect to the total weight of the composition, an Aged Tangerine Peel in an amount of 12 units by weight with respect to the total weight of the composition, a Giant Knotweed in an amount of 25 units by weight with respect to the total weight of the composition, a Reed Rhizome in an amount of 18 units by weight with respect to the total weight of the composition, an Unprepared Licorice Root in an amount of 6 units by weight with respect to the total weight of the composition, and a Fresh Ginger Rhizome in an amount of 6 units by weight with respect to the total weight of the composition. In the present implementation, the above-mentioned proportioned dose is a daily dose, and it should be taken half an hour before the meal in the morning and evening. The duration of usage is 10 days. In this implementation, the main purpose of the composite is to protect the respiratory tract from virus invasion, and the immune function is also enhanced. It is suitable for the prevention of young and middle-aged people.

### Example 3

In another implementation, the composition in the present disclosure includes an Astragalus Root in an amount of 5 units by weight with respect to a total weight of the composition, a Ganoderma Spore in an amount of 2 units by weight with respect to the total weight of the composition, a Siberian Ginseng in an amount of 6 units by weight with respect to the total weight of the composition, an Underleaf Pearl in an amount of 5 units by weight with respect to the total weight of the composition, a Honeysuckle Flower in an amount of 5 units by weight with respect to the total weight of the composition, a Chinese Globe Flower in an amount of 5 units by weight with respect to the total weight of the composition, an Isatis Leaf in an amount of 5 units by weight with respect to the total weight of the composition, an Woad Root in an amount of 6 units by weight with respect to the total weight of the composition, a Patchouli in an amount of 6 units by weight with respect to the total weight of the composition, a Forsythia Fruit in an amount of 6 units by weight with respect to the total weight of the composition, an asiatic Pennywort in an amount of 5 units by weight with respect to the total weight of the composition, an Aged Tangerine Peel in an amount of 3 units by weight with respect to the total weight of the composition, a Giant Knotweed in an amount of 6 units by weight with respect to the total weight of the composition, a Reed Rhizome in an amount of 9 units by weight with respect to the total weight of the composition, an Unprepared Licorice Root in an amount of 2 units by weight with respect to the total weight of the composition, and a Fresh Ginger Rhizome in an amount of 2 units by weight with respect to the total weight of the composition. In the present implementation, the above-mentioned proportioned dose is a daily dose, and it should be taken half an hour before the meal in the morning and evening. The duration of usage is 10 days. It should be taken once a day for children under 10 years old. Infants are not suitable for taking the composition. In this implementation, the main purpose of the composite is to protect the respiratory tract from virus invasion, and the immune function is also enhanced. It is suitable for the prevention of young and middle-aged people. The proportioning portion of these ingredients in the present implementation is lower than the standard dose. It may not only increase the immune function but also protect the respiratory tract from virus invasion. This composition is especially suitable for the prevention of people under 16 years of age.

Clinical Practice Result:

1. In March 2009, the composition in the present disclosure was used to prevent the infection of the influenza A (H1N1) virus in a total of 206 students and faculty members of an art middle school in a certain area.

Among this group of people, there were 182 persons aged 15-17 years old, 22 persons aged 26-55 years, and 2 persons aged 56-60 years, including 77 males and 129 females. The method of prevention is to take Example 3 granules by students under the age of 16 and Example 2 granules orally for 17-60 years of age twice a day for 10 days. As a result, none of the 206 people has influenza A infection.

2. In November 2018-January 2019, the composition in the present disclosure used to prevent and control influenza in a nursing home.

The nursing home has a total of 61 pensioners, including 11 nursing workers. From Oct. 25, 2018, to Nov. 6, 2018, three elderly people died of influenza virus infection and underlying diseases, two of which were influenza A virus, and one was an influenza B virus.

In this group of the experimental population, there are 69 old-age pensioners plus staff, 8 persons aged 60-70 years old, 27 persons aged 71-80 years old, 22 persons aged 81-90 years old, 3 persons aged above 91 years old and 9 persons aged below 60 years old. There were 27 men and 42 women. Starting to take this composition on Nov. 6, 2018, the orally administered granules of Example 1 over the age of 60, and the orally administered granules of Example 2 under the age of 60, twice a day, orally for 10 days. After 45 days of observation, among the 69 cases, there is one case who had a fever, with a maximum body temperature of 38.1 degrees Celsius, with a sore throat, and the body temperature subsided within 3 days after symptomatic treatment. One case died of sudden arrhythmia. There were no other flu symptoms such as fever, throat, and cough among the others. One case of flu symptoms was mild and controlled within a short time.

Plural instances may be provided for components, operations, or structures described herein as a single instance. Finally, boundaries between various components, operations, and data stores are somewhat arbitrary, and particular operations are illustrated in the context of specific illustrative configurations. Other allocations of functionality are envisioned and may fall within the scope of the implementation(s). In general, structures and functionality presented as separate components in the example configurations may be implemented as a combined structure or component. Similarly, structures and functionality presented as a single component may be implemented as separate components. These and other variations, modifications, additions, and improvements fall within the scope of the implementation(s).

It will also be understood that, although the terms “first,” “second,” etc. may be used herein to describe various elements, these elements should not be limited by these terms. These terms are only used to distinguish one element from another. For example, a first column could be termed a second column, and, similarly, a second column could be termed the first column, without changing the meaning of the description, so long as all occurrences of the “first column” are renamed consistently and all occurrences of the “second column” are renamed consistently. The first column and the second are columns both column s, but they are not the same column.

The terminology used herein is for the purpose of describing particular implementations only and is not intended to be limiting of the claims. As used in the description of the implementations and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined (that a stated condition precedent is true)” or “if (a stated condition precedent is true)” or “when (a stated condition precedent is true)” may be construed to mean “upon determining” or “in response to determining” or “in accordance with a determination” or “upon detecting” or “in response to detecting” that the stated condition precedent is true, depending on the context.

The foregoing description included example systems, methods, techniques, instruction sequences, and computing machine program products that embody illustrative implementations. For purposes of explanation, numerous specific details were set forth in order to provide an understanding of various implementations of the inventive subject matter. It will be evident, however, to those skilled in the art that implementations of the inventive subject matter may be practiced without these specific details. In general, well-known instruction instances, protocols, structures, and techniques have not been shown in detail.

The foregoing description, for purpose of explanation, has been described with reference to specific implementations. However, the illustrative discussions above are not intended to be exhaustive or to limit the implementations to the precise forms disclosed. Many modifications and variations are possible in view of the above teachings. The implementations were chosen and described in order to best explain the principles and their practical applications, to thereby enable others skilled in the art to best utilize the implementations and various implementations with various modifications as are suited to the particular use contemplated.

What is claimed is:

1. A composition used for treatment of protecting respiratory tract from viral infection by significantly increasing immunity of a patient, comprising:
an Astragalus Root in an amount from 5 units to 50 units by weight with respect to a total weight of the composition;
a Honeysuckle Flower in an amount from 5 units to 50 units by weight with respect to the total weight of the composition;
an Isatis Leaf in an amount from 5 units to 30 units by weight with respect to the total weight of the composition;
a Woad Root in an amount from 6 units to 50 units by weight with respect to the total weight of the composition;
a Patchouli in an amount from 6 units to 30 units by weight with respect to the total weight of the composition;
a Forsythia Fruit in an amount from 6 units to 30 units by weight with respect to the total weight of the composition;
n Aged Tangerine Peel in an amount from 3 units to 30 units by weight with respect to the total weight of the composition;
a Reed Rhizome in an amount from 9 units to 60 units by weight with respect to the total weight of the composition; and
an Unprepared Licorice Root in an amount from 2 units to 10 units by weight with respect to the total weight of the composition.

2. The composition as claimed in claim 1, wherein
the Astragalus Root is in an amount of 9 units by weight with respect to a total weight of the composition;
the Honeysuckle Flower is in an amount of 5 units by weight with respect to the total weight of the composition;
the Isatis Leaf is in an amount of 5 units by weight with respect to the total weight of the composition;
the Woad Root is in an amount of 5 units by weight with respect to the total weight of the composition;
the Patchouli is in an amount of 6 units by weight with respect to the total weight of the composition;
the Forsythia Fruit is in an amount of 5 units by weight with respect to the total weight of the composition;
the Aged Tangerine Peel is in an amount of 3 units by weight with respect to the total weight of the composition;
the Reed Rhizome is in an amount of 9 units by weight with respect to the total weight of the composition; and
an Unprepared Licorice Root is in an amount of 2 units by weight with respect to the total weight of the composition.

3. A composition used for treatment of protecting respiratory tract from viral infection by significantly increasing immunity of a patient, comprising:
an Astragalus Root in an amount from 5 units to 50 units by weight with respect to a total weight of the composition;
a Honeysuckle Flower in an amount from 5 units to 50 units by weight with respect to the total weight of the composition;
a Woad Root in an amount from 6 units to 50 units by weight with respect to the total weight of the composition;
a Patchouli in an amount from 6 units to 30 units by weight with respect to the total weight of the composition;
a Forsythia Fruit in an amount from 6 units to 30 units by weight with respect to the total weight of the composition;
an Aged Tangerine Peel in an amount from 3 units to 30 units by weight with respect to the total weight of the composition;
a Giant Knotweed in an amount from 6 units to 50 units by weight with respect to the total weight of the composition;
a Reed Rhizome in an amount from 9 units to 60 units by weight with respect to the total weight of the composition;
an Unprepared Licorice Root in an amount from 2 units to 10 units by weight with respect to the total weight of the composition; and
a Fresh Ginger Rhizome in an amount from 2 units to 15 units by weight with respect to the total weight of the composition.

4. The composition as claimed in claim 3, wherein
the Astragalus Root is in an amount of 9 units by weight with respect to a total weight of the composition;
the Honeysuckle Flower is in an amount of 6 units by weight with respect to the total weight of the composition;
the Woad Root is in an amount of 8 units by weight with respect to the total weight of the composition;
the Patchouli is in an amount of 6 units by weight with respect to the total weight of the composition;
the Forsythia Fruit is in an amount of 6 units by weight with respect to the total weight of the composition;
the Aged Tangerine Peel is in an amount of 6 units by weight with respect to the total weight of the composition;
the Giant Knotweed is in an amount of 5 units by weight with respect to the total weight of the composition;
the Reed Rhizome is in an amount of 8 units by weight with respect to the total weight of the composition;
the Unprepared Licorice Root is in an amount of 3 units by weight with respect to the total weight of the composition, and
the Fresh Ginger Rhizome is in an amount of 3 units by weight with respect to the total weight of the composition.

5. A composition used for treatment of protecting respiratory tract from viral infection by significantly increasing immunity of a patient, comprising:
an Astragalus Root in an amount from 5 units to 50 units by weight with respect to a total weight of the composition;
a Ganoderma Spore in an amount from 2 units to 21 units by weight with respect to the total weight of the composition;
a Siberian Ginseng in an amount from 6 units to 30 units by weight with respect to the total weight of the composition;
an Underleaf Pearl in an amount from 5 units to 35 units by weight with respect to the total weight of the composition;
a Honeysuckle Flower in an amount from 5 units to 50 units by weight with respect to the total weight of the composition;
n Woad Root in an amount from 6 units to 50 units by weight with respect to the total weight of the composition;
a Patchouli in an amount from 6 units to 30 units by weight with respect to the total weight of the composition;
a Forsythia Fruit in an amount from 6 units to 30 units by weight with respect to the total weight of the composition;
an Aged Tangerine Peel in an amount from 3 units to 30 units by weight with respect to the total weight of the composition;
a Reed Rhizome in an amount from 8 units to 60 units by weight with respect to the total weight of the composition;
an Unprepared Licorice Root in an amount from 2 units to 10 units by weight with respect to the total weight of the composition; and
a Fresh Ginger Rhizome in an amount from 2 units to 15 units by weight with respect to the total weight of the composition.

6. The composition as claimed in claim 5, wherein
the Astragalus Root is in an amount of 9 units by weight with respect to a total weight of the composition;
the Ganoderma Spore is in an amount of 2 units by weight with respect to the total weight of the composition;
the Siberian Ginseng is in an amount of 6 units by weight with respect to the total weight of the composition;
the Underleaf Pearl is in an amount of 6 units by weight with respect to the total weight of the composition;
the Honeysuckle Flower is in an amount of 6 units by weight with respect to the total weight of the composition;
the Woad Root is in an amount of 8 units by weight with respect to the total weight of the composition;
the Patchouli is in an amount of 6 units by weight with respect to the total weight of the composition;
the Forsythia Fruit is in an amount of 6 units by weight with respect to the total weight of the composition;
the Aged Tangerine Peel is in an amount of 6 units by weight with respect to the total weight of the composition;
the Reed Rhizome is in an amount of 8 units by weight with respect to the total weight of the composition;
the Unprepared Licorice Root is in an amount of 3 units by weight with respect to the total weight of the composition; and
the Fresh Ginger Rhizome is in an amount of 3 units by weight with respect to the total weight of the composition.

7. A composition used for treatment of protecting respiratory tract from viral infection by significantly increasing immunity of a patient, comprising:
an Astragalus Root in an amount from 5 units to 50 units by weight with respect to a total weight of the composition;
a Ganoderma Spore in an amount from 2 units to 21 units by weight with respect to the total weight of the composition;
a Siberian Ginseng in an amount from 6 units to 30 units by weight with respect to the total weight of the composition;
an Underleaf Pearl in an amount from 5 units to 35 units by weight with respect to the total weight of the composition;
a Honeysuckle Flower in an amount from 5 units to 50 units by weight with respect to the total weight of the composition;
a Chinese Globe Flower in an amount from 5 units to 30 units by weight with respect to the total weight of the composition;
an Isatis Leaf in an amount from 5 units to 30 units by weight with respect to the total weight of the composition;
a Woad Root in an amount from 6 units to 50 units by weight with respect to the total weight of the composition;
a Patchouli in an amount from 6 units to 30 units by weight with respect to the total weight of the composition;
a Forsythia Fruit in an amount from 6 units to 30 units by weight with respect to the total weight of the composition;
an asiatic Pennywort in an amount from 5 units to 50 units by weight with respect to the total weight of the composition;
an Aged Tangerine Peel in an amount from 3 units to 30 units by weight with respect to the total weight of the composition;
a Giant Knotweed in an amount from 6 units to 50 units by weight with respect to the total weight of the composition;
a Reed Rhizome in an amount from 9 units to 60 units by weight with respect to the total weight of the composition;
an Unprepared Licorice Root in an amount from 2 units to 10 units by weight with respect to the total weight of the composition; and
a Fresh Ginger Rhizome in an amount from 2 units to 15 units by weight with respect to the total weight of the composition.

8. The composition as claimed in claim 7, wherein
the Astragalus Root is in an amount of 15 units by weight with respect to a total weight of the composition;
the Ganoderma Spore is in an amount of 3 units by weight with respect to the total weight of the composition;
the Siberian Ginseng is in an amount of 10 units by weight with respect to the total weight of the composition;
the Underleaf Pearl is in an amount of 10 units by weight with respect to the total weight of the composition;
the Honeysuckle Flower is in an amount of 12 units by weight with respect to the total weight of the composition;
the Chinese Globe Flower is in an amount of 10 units by weight with respect to the total weight of the composition;
the Isatis Leaf is in an amount of 10 units by weight with respect to the total weight of the composition;
the Woad Root is in an amount of 10 units by weight with respect to the total weight of the composition;
the Patchouli is in an amount of 10 units by weight with respect to the total weight of the composition;
the Forsythia Fruit is in an amount of 10 units by weight with respect to the total weight of the composition;
the asiatic Pennywort is in an amount of 10 units by weight with respect to the total weight of the composition;
the Aged Tangerine Peel is in an amount of 10 units by weight with respect to the total weight of the composition;
the Giant Knotweed is in an amount of 15 units by weight with respect to the total weight of the composition;
the Reed Rhizome is in an amount of 15 units by weight with respect to the total weight of the composition;
the Unprepared Licorice Root is in an amount of 3 units by weight with respect to the total weight of the composition; and
the Fresh Ginger Rhizome in an amount of 3 units by weight with respect to the total weight of the composition.

9. The composition used for treatment of protecting respiratory tract from viral infection by significantly increasing immunity of a patient as claimed in claim 7, wherein:
the Astragalus Root is in an amount of 25 units by weight with respect to the total weight of the composition;
the Ganoderma Spore is in an amount of 5 units by weight with respect to the total weight of the composition;
the Siberian Ginseng is in an amount of 15 units by weight with respect to the total weight of the composition;
the Underleaf Pearl is in an amount of 12 units by weight with respect to the total weight of the composition;
the Honeysuckle Flower is in an amount of 15 units by weight with respect to the total weight of the composition;
the Chinese Globe Flower is in an amount of 10 units by weight with respect to the total weight of the composition;
the Isatis Leaf is in an amount of 10 units by weight with respect to the total weight of the composition;
the Woad Root is in an amount of 15 units by weight with respect to the total weight of the composition;
the Patchouli is in an amount of 10 units by weight with respect to the total weight of the composition;
the Forsythia Fruit is in an amount of 10 units by weight with respect to the total weight of the composition;
the asiatic Pennywort is in an amount of 10 units by weight with respect to the total weight of the composition;
the Aged Tangerine Peel is in an amount of 10 units by weight with respect to the total weight of the composition;
the Giant Knotweed is in an amount of 15 units by weight with respect to the total weight of the composition;
the Reed Rhizome is in an amount of 15 units by weight with respect to the total weight of the composition;
the Unprepared Licorice Root is in an amount of 6 units by weight with respect to the total weight of the composition; and
the Fresh Ginger Rhizome is in an amount of 6 units by weight with respect to the total weight of the composition.

10. The composition used for treatment of protecting respiratory tract from viral infection by significantly increasing immunity of a patient as claimed in claim 7, wherein:
the Astragalus Root is in an amount of 10 units by weight with respect to the total weight of the composition;
the Ganoderma Spore is in an amount of 3 units by weight with respect to the total weight of the composition;
the Siberian Ginseng is in an amount of 10 units by weight with respect to the total weight of the composition;
the Underleaf Pearl is in an amount of 12 units by weight with respect to the total weight of the composition;
the Honeysuckle Flower is in an amount of 18 units by weight with respect to the total weight of the composition;
the Chinese Globe Flower is in an amount of 15 units by weight with respect to the total weight of the composition;
the Isatis Leaf is in an amount of 18 units by weight with respect to the total weight of the composition;
the Woad Root is in an amount of 30 units by weight with respect to the total weight of the composition;
the Patchouli is in an amount of 12 units by weight with respect to the total weight of the composition;
the Forsythia Fruit is in an amount of 12 units by weight with respect to the total weight of the composition;
the asiatic Pennywort is in an amount of 12 units by weight with respect to the total weight of the composition;
the Aged Tangerine Peel is in an amount of 12 units by weight with respect to the total weight of the composition;
the Giant Knotweed is in an amount of 25 units by weight with respect to the total weight of the composition;
the Reed Rhizome is in an amount of 18 units by weight with respect to the total weight of the composition;
the Unprepared Licorice Root is in an amount of 6 units by weight with respect to the total weight of the composition; and
the Fresh Ginger Rhizome is in an amount of 6 units by weight with respect to the total weight of the composition.

11. The composition used for treatment of protecting respiratory tract from viral infection by significantly increasing immunity of a patient as claimed in claim 7, wherein:
the Astragalus is in an amount of 5 units by weight with respect to the total weight of the composition;
the Ganoderma Spore is in an amount of 2 units by weight with respect to the total weight of the composition;
the Siberian Ginseng is in an amount of 6 units by weight with respect to the total weight of the composition;
the Underleaf Pearl is in an amount of 5 units by weight with respect to the total weight of the composition;
the Honeysuckle Flower is in an amount of 5 units by weight with respect to the total weight of the composition;
the Chinese Globe Flower is in an amount of 5 units by weight with respect to the total weight of the composition;
the Isatis Leaf is in an amount of 5 units by weight with respect to the total weight of the composition;
the Woad Root is in an amount of 6 units by weight with respect to the total weight of the composition;
the Patchouli is in an amount of 6 units by weight with respect to the total weight of the composition;
the Forsythia Fruit is in an amount of 6 units by weight with respect to the total weight of the composition;
the asiatic Pennywort is in an amount of 5 units by weight with respect to the total weight of the composition;
the Aged Tangerine Peel is in an amount of 3 units by weight with respect to the total weight of the composition;
the Giant Knotweed is in an amount of 6 units by weight with respect to the total weight of the composition;
the Reed Rhizome is in an amount of 9 units by weight with respect to the total weight of the composition;
the Unprepared Licorice Root is in an amount of 2 units by weight with respect to the total weight of the composition; and
the Fresh Ginger Rhizome is in an amount of 2 units by weight with respect to the total weight of the composition.
